(19) United States
(12) Patent Application Publication
(10) Pub. No.: US 2007/0190175 A1
(43) Pub. Date: Aug. 16, 2007

(54) SKIN CARE COMPOSITION FOR DERMATOLOGICAL DISORDERS

(75) Inventors: Barry W. Cummins, Fort Pierce, FL (US); David H. Creasey, Cocoa, FL (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(73) Assignee: Tasker Products IP Holding Corp

(21) Appl. No.: 11/608,669

(22) Filed: Dec. 8, 2006

Related U.S. Application Data
(60) Provisional application No. 60/748,692, filed on Dec. 8, 2005.

(51) Int. Cl. A61K 33/34; A61K 33/06; A61K 33/38

(52) U.S. Cl. 424/618; 424/630; 424/637; 424/641; 424/682

(57) ABSTRACT

A topical skin care composition is provided to improve skin texture, diminish fine lines and wrinkles and decrease the appearance of hyper-pigmented areas. In addition, another embodiment of the skin care composition can be used to treat burns, insect bites and diminish the pain and scarring caused by such injuries.

### CROSS-REFERENCE TO RELATED APPLICATIONS

This application claims priority from U.S. Provisional Application Ser. No. 60/748,692 filed on Dec. 8, 2005, which is incorporated herein by reference in its entirety.

### FIELD OF THE INVENTION

This invention relates to a skin care composition and in particular to a composition, a method of making and using the composition as a topical application for dermatological disorders including wrinkles, burns, insect bites and the like.

### BACKGROUND

A wide variety of compositions are known for providing cosmetic and/or pharmacologic benefits to human skin. The development of effective emollients, liquids, creams, or sprays to modify or restore skin textual differences that are caused by injuries, sun damage and aging has been the focus of cosmetologists and dermatologists in recent years.

The most widely studied topical products are retinoids, hydroxy acids and vitamins A, C and E. Recently, metallic ions have been used in animal studies to heal wounds and stimulate collagen. In clinical trials, copper and other metallic ions have outperformed the more established rivals, including creams and lotions containing vitamin C, melatonin and retinoid drugs. Simeon et al. have reported on the use of tripeptide copper complexes to promote wound healing in the J1 Invest Dermatol (1999), Vol. 112, 957-964; Life Sci (2000), Vol. 67, 2257-2265 and JI Invest Dermatol (2000), Vol. 115, 962-968. Maquart et al. discussed the use of tri-peptide-copper complexes to increase collagen synthesis in FEBS Letters (1988), Vol. 238, 343-346 and JI Clin Invest (1993), Vol. 92.2368-2376.

Trace amounts of metals or metallic ions have been recognized as essential to human health for a long time. Copper bangles have been worn since ancient times to keep rheumatism at bay. In 1928, a study showed that copper, the third most abundant trace metal in our bodies after iron and zinc, helps prevent anemia. Magnesium helps the body metabolize calcium and build stronger bones. Silver ions are known for antibacterial activity, which is an important function in healing wounds.

Unfortunately, the human body cannot synthesize any of the metallic ions. The typical Western diet is low in metallic ions, which are found in legumes, crab, wheat germ and other unprocessed foods. Most of what we do consume goes to the vital organs, and very little makes its way to the epidermis.

Scientists have spent the past decade trying to work out how to deliver metallic ions, most recently, copper ions, directly to the skin. This is not as easy as it sounds because the copper molecules react uselessly with traditional ingredients in skin creams and in many of the existing skin care preparations containing copper the copper is not delivered to the skin cells in a usable form.

There is a need for a composition that can effectively deliver metallic ions to the epidermis in a usable form. The present invention provides an effective delivery system for metallic ions to desired locations in human epidermis.

In U.S. Pat. Nos. 5,989,595 and 6,242,011 BI to Cummins, an acidic composition of matter is disclosed that is useful for destroying microorganisms that spoil food, such as fish. The composition of matter, patented by Cummins, is also useful for skin treatment of melanoma and the treatment of other bacteria, and serves as the precursor for the novel skin care composition disclosed herein.

### SUMMARY OF THE INVENTION

The first objective of the present invention is to provide a skin care composition that decreases superficial wrinkles commonly associated with photoaging of human skin.

The second objective of the present invention is to provide a skin care composition that reduces hyper-pigmented areas commonly associated with photoaging of human skin.

The third objective of the present invention is to provide a skin care composition that reduces textural changes of the face and perioral region that surrounds the entrance to the oral cavity, commonly associate with photoaging of human skin.

The fourth objective of the present invention is to provide a skin care composition that promotes the healing of wounds.

The fifth objective of the present invention is to provide a skin care composition that effectively treats sunburns, and skin burns from excessive heat or radiation. including UV radiation from the sun.

The sixth objective of the present invention is to provide a skin care composition that effectively reduces burn scars.

The seventh objective of the present invention is to provide a topical skin care composition that effectively delivers copper to the epidermis.

The eighth objective of the present invention is to provide a topical skin care composition that effectively delivers zinc to the epidermis.

The ninth objective of the present invention is to provide a topical skin care composition that effectively delivers silver to the epidermis.

The tenth objective of the present invention is to provide a topical skin care composition that effectively delivers magnesium to the epidermis.

A preferred anti-wrinkle skin care composition is provided when an effective amount of PHB0020 with uniformly suspended metallic ions is mixed with a standard dermal cream and water to form a cream for use on the skin of warm-blooded animals, including humans.

A more preferred anti-wrinkle skin care composition contains metallic ions, such as copper, magnesium, silver and zinc; most preferably copper ions.

A preferred method for treating, preventing or ameliorating environmental or age related damage or deterioration of the skin includes applying a skin care effective amount of the skin care preparation of the present invention on a daily basis for a period of from approximately two weeks to approximately ten weeks.

The composition of the present invention can be in the form of a paste, gel, cream or liquid.

The composition of the present invention can be used to facilitate the healing of burns to the skin, such that scarring is minimal.

Other objects and features will be in part apparent and in part pointed out hereinafter.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1 is a graph showing the effect of PHB0020 on pathogenic and spoilage bacterial isolates exposed for 2 minutes.

FIG. 2 is a graph showing the logarithm of reductions in bacterial colony levels.

### DETAILED DESCRIPTION OF THE INVENTION

Before explaining the disclosed embodiment of the present invention in detail, it is to be understood that the invention is not limited in its application to the details of the particular arrangement shown since the invention is capable of other embodiments. Also, the terminology used herein is for the purpose of description and not of limitation.

It would be useful to discuss the meanings of some words used herein and their applications before discussing the composition of matter and method of using and making the same. The following definitions and methods are provided to better define the present invention and to guide those of ordinary skill in the art in the practice of the present invention. Unless otherwise noted, terms are to be understood according to conventional usage by those of ordinary skill in the relevant art.

As used herein, the term “PHB0020” refers to copper sulfate pentahydrate and/or other forms of copper ions, and silver sulfate and/or other forms of silver ions added to pHarlo for the antimicrobial, anti-bacterial additive of the present invention.

As used herein, the term “pHarlo” refers to a composition of matter claimed in U.S. Pat. Nos. 5,989,595 and 6,242,001 B1 to Cummins and incorporated herein by reference and more completely described below.

Salmonella refers to Salmonella typhimurium, a pathogen. Listeria refers to Listeria monocvtogenes, a pathogen. Staph refers to Staphylococcus aureus, a pathogen. E-coli refers to Escherichia coli, an indicator bacteria. Pseudomonas refers to Pseudomonas fluorescens, a spoilage bacteria. Shewanella refers to Shewanella putrefaciens, a spoilage bacteria.

Percent by weight means the weight of the ingredient per weight of the overall 20 formulation and is abbreviated herein as “Percent W/W”.

Various formulations of a skin care composition have been provided and the world of cosmetology and dermatology are the beneficiaries of a composition that improves skin texture, diminishes fine lines and wrinkles, decreases the appearance of hyper pigmented areas, heals skin burns, diminishes the painful effects of sun bur and insect bites, reduces scarring from burs and insect bites.

The present invention includes a skin care composition that decreases superficial wrinkles commonly associated with photoaging of human skin. The present invention also includes skin care compositions that reduces hyper-pigmented areas commonly associated with photoaging of human skin; reduces textural changes of the face and perioral region that surrounds the entrance to the oral cavity, commonly associate with photoaging of human skin; promotes the healing of wounds; effectively treats sunburns, and skin burns from excessive heat or radiation, including UV radiation from the sun; reduces burn scars; delivers metallic ions such as copper, zinc, silver, and/or magnesium to the epidermis.

A preferred anti-wrinkle skin care composition is comprises an effective amount of PHB0020 with uniformly suspended metallic ions mixed with a standard dermal cream and water to form a cream for use on the skin of warm-blooded animals, including humans. A more preferred anti-wrinkle skin care composition contains metallic ions, such as copper, magnesium, silver and zinc; most preferably copper ions.

The invention provides a preferred method for treating, preventing or ameliorating environmental or age related damage or deterioration of the skin that includes applying a skin care effective amount of the skin care preparation of the present invention on a daily basis for a period of from approximately two weeks to approximately ten weeks.

The composition of the present invention can be in the form of a paste, gel, cream or liquid. Formulation of such is known to those skilled in the art. The agents described herein can be formulated by any conventional manner using one or more acceptable carriers and/or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of the agent, together with a suitable amount of carrier so as to provide the form for proper administration to the subject.

The composition of the present invention can be used to facilitate the healing of burns to the skin, such that scarring is minimal.

Referring now to the composition of pHarlo Blue 0020, hereinafter referred to as PHB0020, it is an antimicrobial, anti-bacterial agent which has a formulation that is generally recognized as safe (GRAS) by the US Food and Drug Administration. One embodiment of the composition is listed below:

| Ingredient | Percentage |
|---|---|
| Copper Sulfate | 16.4 |
| Pentahydrate | |
| Sulfuric Acide | 9.9 |
| (processing aid | |
| Ammonium sulfate | 2.2 |
| Distilled water | 71.5 |

The ingredients form a concentrate, which can be combined in small amounts of less than 0.10 milliliters (ml) with 1 gallon of water to make PHB0020. Examples described herein provide greater detail on the use and effectiveness of PHB0020.

Having described the invention in detail, it will be apparent that modifications, variations, and equivalent embodiments are possible without departing the scope of the invention defined in the appended claims. Furthermore, it should be appreciated that all examples in the present disclosure are provided as non-limiting examples.

### EXAMPLES

The following non-limiting examples are provided to further illustrate the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

### pHarlo Preparation

First, a pressurized vessel is selected that includes a cooling jacket and no electrode attachments: however, the preferred pressurized vessel is fitted with two electrodes, a cathode and anode, to provide a direct current (DC) voltage one (1) foot above the bottom of the container. The electrodes are spaced approximately three (3) feet apart.

Preferable processing steps of the present invention can include combining sulfuric acid with purity in a range from approximately 94% to approximately 99.9% in a 1 to 2 volume ratio with distilled water and ammonium sulfate in a ratio of 2.77 pounds of ammonium sulfate per gallon of distilled water to provide mixture (I). The mixture (I) is combined in a pressurized vessel having preferably two strategically placed electrodes, a cathode and anode. During the addition of ammonium sulfate, a direct current (DC) voltage is applied to the mixture. The voltage is applied in a range from approximately one (1) amp to approximately 100 amps, preferably between approximately 1 amp and approximately 5 amps. The mixture is then heated under pressure in a range of from approximately 1 pound per square inch (psi) to approximately 15 psi above atmospheric pressure. Heating of the mixture is in a range of from approximately 200° Fahrenheit (F) to approximately 1200° F. preferably from approximately 800° F. to approximately 900° F. for approximately 30 minutes. With the application of heat and pressure as specified above, it is understood by persons skilled in the art, that a judicious selection of temperature, time and pressure is required and should be adjusted to maintain a safe chemical reaction.

After cooling the mixture, a stabilizer is added. The stabilizer is a portion of mixture (I) prior to heating in the pressure vessel. The quantity of stabilizer used is approximately 10 weight percent of the total weight of mixture (I). The resulting acidic composition is useful for destroying microorganisms having a pH of negative 3 (−3). To the resulting acidic composition can be added compounds containing metallic ions for the extensive properties, including but not limited to antimicrobial properties, discussed herein. The following physical and chemical properties are observed when undiluted.

The pH is about −3, which was determined by a non acidified hydrogen proton count with the data corrected for any electrode type errors, and was performed by EFE&H analytical services, an EPA (Environmental Protection Agency) approved laboratory. The metallic ions demonstrated stability in solution from approximately 0 pH up to approximately 9 pH. The metallic ions demonstrated stability in solution with temperature of from approximately 32° F. to the point of vaporization, or approximately 212° F.

Various other compounds with metallic ions may be substituted for copper sulfate pentahydrate. The following metal salts are suitable substitutes: Copper sulfate, copper glutamate, zinc oxide, zinc glutamate, magnesium glutamate, magnesium sulfate, silver sulfate, silver oxide, and combinations thereof.

### Example 2

### Formulations

The inhibitory activity of acidic buffered disinfection agents on aerobic plate count (APC) was examined. Five formulations were tested.

Mark I: a 24 hour high temperature reaction process at approximately 300-350° F. with a stabilization step after overnight cooling. Composed of reacting 98% sulfuric acid with a 26-28% by weight ammonium sulfate in water solution. The order of addition was ammonium sulfate solution to sulfuric acid. Electrolysis of the reacting solution was applied for 1 hour at the start of the process. The stabilization step was the addition of more ammonium sulfate solution to ensure that the reaction is complete. The Tasker Clear™ product formed was a buffered acid solution of a strong acid (sulfuric acid) and a salt (ammonium sulfate) of a strong acid and strong base.

Mark II: a 2 hour low temperature reaction process at approximately 200-210° F. with a stabilization step immediately after the 1 hour electrolysis period. This was the same process as in the Mark I product above except that it was performed at a lower temperature and a shorter period of time. The ingredient amounts were adjusted to account for no lost of water as was seen in the Mark I process. The Tasker Clear™ product formed was a buffered acid solution of a strong acid (sulfuric acid) and a salt (ammonium sulfate) of a strong acid and strong base.

Mark III: a low temperature reaction process in which the 98% sulfuric acid was added slowly to a 30% by weight ammonium sulfate solution. The addition was done continuously until all the ammonium sulfate solution was added. There was no stabilization step. The addition order was the reverse of the Mark I, II, IV, and V processes. The temperature was maintained in the 150-200° F. range during the addition process. No electrolysis was performed during this process and hence the name ‘cold process’ was given to it. The Tasker Clear™ product formed was a buffered acid solution of a strong acid (sulfuric acid) and a salt (ammonium sulfate) of a strong acid and strong base.

Mark IV: a 4 hour high temperature reaction process at approximately 300-350° F. with a stabilization step after cooling. Composed of reacting 98% sulfuric acid with a 26-28% by weight sodium sulfate in water solution. The order of addition was sodium sulfate solution to sulfuric acid. Electrolysis of the reacting solution was applied for 1 hour at the start of the process. The stabilization step was the addition of more sodium sulfate solution to ensure that the reaction is complete. The Tasker Clear™ product formed was a buffered acid solution of a strong acid (sulfuric acid) and a salt (sodium sulfate) of a strong acid and strong base. (Note: In this process sodium sulfate was substituted for ammonium sulfate.)

Mark V: a 4 hour high temperature reaction process at approximately 300-350° F. with a stabilization step after cooling. Composed of reacting 98% sulfuric acid with a 26-28% by weight sodium sulfate in water solution. The order of addition was sodium sulfate solution to sulfuric acid. There was no electrolysis during this process (cold process). The stabilization step was the addition of more sodium sulfate solution to ensure that the reaction was complete. The Tasker Clear™ product formed was a buffered acid solution of a strong acid (sulfuric acid) and a salt (sodium sulfate) of a strong acid and strong base. (Note: In this process sodium sulfate was substituted for ammonium sulfate, and no electrolysis was performed.)

Results showed that all formulations exponentially reduced the aerobic plate count (see e.g., Table 1).

TABLE 1

| | | |
|---|---|---|
| | Counts | Log₁₀ |
| Time | cfu/ml | cfu/ml |

Butterfield Buffer Control

| | | |
|---|---|---|
| 0 | 845 | 2.93 |
| 5 | 780 | 2.89 |
| 15 | 785 | 2.89 |
| | | Ave = 2.90 |

DI Water Control

| | | |
|---|---|---|
| 0 | 1015 | 3.01 |
| 5 | 1075 | 3.03 |
| 15 | 940 | 2.97 |
| | | Ave = 3.00 |

| | | | |
|---|---|---|---|
| | Counts | Log₁₀ | Log |
| Time | cfu/ml | cfu/ml | Reduction |

Mark I Solution

| | | | |
|---|---|---|---|
| 0 | 140 | 2.15 | 0.85 |
| 5 | 25 | 1.40 | 1.60 |
| 15 | 5 | 0.70 | 2.30 |

Mark II Solution

| | | | |
|---|---|---|---|
| 0 | 100 | 2.00 | 1.00 |
| 5 | 30 | 1.48 | 1.52 |
| 15 | 0 | 0.00 | 3.00 |

Mark III Solution

| | | | |
|---|---|---|---|
| 0 | 65 | 1.81 | 1.19 |
| 5 | 0 | 0.00 | 3.00 |
| 15 | 0 | 0.00 | 3.00 |

Mark IV Solution

| | | | |
|---|---|---|---|
| 0 | 110 | 2.04 | 0.96 |
| 5 | 40 | 1.60 | 1.40 |
| 15 | 0 | 0.00 | 3.00 |

Mark V Solution

| | | | |
|---|---|---|---|
| 0 | 125 | 2.10 | 0.90 |
| 5 | 20 | 1.30 | 1.70 |
| 15 | 5 | 0.70 | 2.30 |

NOTES:
* Log Reduction based on DI Water average log₁₀ = 3.00
** Counts are the average of duplicate APC plates

### Example 3

### Dermatological Formulations

In the following examples, certain dermatological disorders are studied to show the efficacy of the delivery of copper and other metallic ions to the epidermis. Although the examples are based on copper ions, this is not a limitation of the present invention and other metallic ions are capable of the same efficient delivery and a person skilled in the art would know which metallic ions would be most effective for a specific dermatological disorder.

TABLE 2
Use Levels in Parts Per Million (ppm):

| Application for PHB0020: | Range | Target |
|---|---|---|
| Anti-wrinkle preparation and | .8 to 2.5 ppm | 1.5 ppm |
| treatment for skin discoloration | | |
| Relief for burns to the skin | 1.2 to 5 ppm | 2.4 ppm |
| Insect bites | 0.5 to 2 ppm | 1.2 ppm |

Three embodiments of the present invention, hereinafter referred to as skin care composition Formulas A, B, and C are prepared at room temperature using the formulations in Table 3. It is understood that the percentage of ingredients for each composition is stated in the most preferred range, while a person skilled in the art would know that the amount of each ingredient can range from 1% to 5% of the amount stated and still provide an effective skin care composition.

Generally, the skin care compositions of the present invention are prepared by first mixing PHB0020, which contains metallic ions, with deionized water and then adding the balance of ingredients, as described above. The ingredients are mixed, for example, in the percentages given in Table 3, as required for each targeted skin treatment. The mixture is thoroughly stirred until the metallic ions in the PHB0020 starting material are completely blended and uniformly suspended.

TABLE 3

| Formulations | PHB0020 content | Cream |
|---|---|---|
| A | 2.6% PHB0020 | 97.4% Water Soluble Dermal |
| | | Base Cream |
| B | 5.4% PHB0020 | 94.6% Oil/water emulsion |
| | | Dermal Base Cream |
| C | 1.5% PHB0020 | 98.5% Glycerin Water Base |
| | | Lotion Cream |

The ingredients of formulation A are mixed to form a topical dermal cream with moisturizing ingredients, collagen, elastin and copper ions in uniform suspension. The pH range for the final formulation is in a range between approximately 2.75 and 3.20, preferable 3.0. The lower, more acidic pH value is responsible for facilitating the absorption of copper ions by the skin or subdermal layer so that the repairs to the skin structures are achieved and/or wrinkles become less visible. The repairs to the subdermal layer also lead to a clearer skin color, eliminating, for example, blotching from exposure to sun and environmental damage for any color skin.

The ingredients of formulation B are mixed to form a lotion or a cream that can be applied to skin that has been burned, for example by heat, radiation from cancer treatment, and/or ultra-violet (LTV) radiation from the sun. The pH range for the final formulation is in a range from approximately 1.4 to approximately 3.4. The skin burn preparation of the present invention works because of highly charged particles in the low pH adjusted preparation that contributes to controlling the burning sensation.

The ingredients of formulation Care mixed to form a lotion, cream, or spray that can be applied to relieve the pain and discomfort of insect bites, such as from fire ants, bees, wasps and the like. The pH range of the final product is adjusted to a range of between approximately 0.5 to approximately 4.0.

A placebo or control was prepared at room temperature and was a mixture of ingredients similar to those of the compositions of the present invention; however, the control does not contain PHB0020.

### Example 4

### Anti-Wrinkle Studies

Current assessments of skin texture, including wrinkles and pigmentation, depend on standardized photographs and various methods of surface proflometry. Proflometry techniques evaluate texture more accurately because these methods reflect the depth and width of wrinkles. However. proflometry methods are difficult to perform and thus are limited to specialized research centers. Using standardized reference photographs, Lemperle developed a wrinkle assessment scale that is easy to use, consistent and correlated well with profilometry measurements. Optical Coherence Tomography (OCT) is a new non-invasive imaging technique that offers a simple method to evaluate the superficial aspects of the skin. OCT uses wavelengths of light (850-1310 nm) that have no known detrimental biological effects. OCT power levels are well below the American National Standards Institute (ANSI) threshold for tissue damage (2-4 mW). OCT not only provides topographical information about the skin surface, it also precisely identifies epidermal thickness and ultrastructural details that reflect skin histology. A dark band in the striatum corneum in OCT images of the skin was found to be 100% specific for actinic keratosis. Actinic keratosis is an overgrowth of skin layers resulting from extended exposure to the sun. The growths begin as flat scaly areas that later develop a hard wart-like surface. Although OCT is a new technology, it is likely that OCT will be increasingly used as an adjunct to the assessment of skin texture and actinic changes caused by radiation. It is also conceivable that OCT may surpass current methods in the validation of cosmetic claims.

Wrinkle assessments and skin discolorations were evaluated using standardized photographs and optical coherence tomograms (OCT).

Thirty healthy female subjects participated in the study. Fifteen subjects were Caucasian and 15 subjects were non-Caucasian, such as, African American, Asian, Hispanic or Philippine ethnicity. The age range for the subjects is between approximately 30 and approximately 60 years. The skin of each subject is graded at baseline using a 0-5 grading scale (0 being normal skin). Subjects eligible for participation have a grade of 2 or greater in both facial fine line/wrinkle and skin texture in the cheek area. Wrinkles are graded according to the criteria published by Lemperle et al. in Plastic Reconstructive Surgery (2001) Vol. 108, 1735-1750. The definition of skin texture includes enlarged pores and pebbly appearance. At least half of the patients also have hyper pigmented regions and/or spotty skin pigmentation in the form of actinic lentigines.

Prior to the study there was a two week washout period in which subjects were instructed to discontinue their normal facial cleanser and moisturizer products for the duration of the study. Each subject was supplied with a commercially available mild facial cleanser, such as Neutrogena liquid™ soap, distributed by Johnson & Johnson, New Brunswick, N.J. 08933. Upon initiation of the study, each subject continued to use the mild facial cleanser and was given two bottles of emollient, one contained the product of the present invention. Formula A and the other bottle contained a placebo or control. The subjects were instructed to use one bottle for the left and the other for the right side of the face. Both the subject and the investigator are blind with respect to the identity of the product of the present invention and the vehicle control or placebo.

Wrinkle Assessments

Two non-invasive methods are used to evaluate the effect of the product of the present invention on skin texture/wrinkles. Standardized photographs and optical coherence tomograms (OCT) of the lateral aspect outer canthus, perioral region and mid face will be made. Baseline and 2 week interval images were obtained over a ten week period.

Wrinkle assessments are made following a blinded protocol with at least two professionals evaluating standardized photographs. Using the protocol published by Gottfried et al., wrinkles are classified from 0 to 5 by correlating the subject photographs to reference standards. All photographs are made at a 1:1 magnification using a dental camera and color corrected light. An American Board of Forensic Odontology (ABFO) photomacrographic scale is placed at the lateral and inferior aspect of the image. The ABFO scale is used to compensate for distortion occurring between photographs; it also has an 18% gray reflectance such that color corrections can be made. Using the surface proflometry capability of OCT the precise volume of surface wrinkles are determined for sequential OCT scans using. To facilitate repositioning of the OCT probe a linear ABFO scale with window for the OCT probe is aligned with the outer canthus and superior tragus for the periorbital lines; the labial commissure and superior-most aspect of the vermillion border for perioral lines and along the ala-tragus line for cheek folds. Three OCT images is made and the average wrinkle volume recorded. Assessments of skin hydration at the OCT imaging sites are determined using a Corneometer (Courage-Khazaka Products, Inc).

The protocol consists of a double-blind placebo-controlled split-face study with left-right randomization. Subjects are evaluated at baseline 2, 4, 6, 8, and 10 weeks. The results of this study will show that the product of the present invention improves skin texture, diminishes fine lines and wrinkles, and improves skin appearance.

### Example 5

### Skin Discoloration Study

In addition to over-all clarity as described in Example 2, assessments of localized skin discoloration will be made in at least half of the subjects. These discolorations include actinic and age-related hyper pigmentation, or melasma. Again, photographs and OCT image are evaluated by at least two professionals at two week intervals. Consistency of skin color and overall skin clarity is assessed following a blinded protocol with at least two professionals evaluating standardized photographs of the mid-face.

The protocol consists of a double-blind placebo-controlled split-face study with left-right randomization. Each subject is instructed to apply daily the product of the present invention, Formula A, and/or the placebo. Subjects are evaluated at baseline 2, 4, 6, 8, and 10 weeks. The results of this study will show that the product of the present invention improves skin texture, reduces hyper pigmentation, and improves skin appearance by minimizing skin tone mottling.

### Example 6

### Effects of Production of H₂0₂, O₂⁻, and Lipid Peroxides

The following example evaluates the effect of formulations of the invention on the production of H₂0₂, O₂⁻, and lipid peroxides in human cell culture exposed to UV radiation through fluorescence cytometry analysis.

The present assay allows the relative quantification of the amount of hydrogen peroxide (H₂0₂), anion superoxide (O₂⁻), and lipid peroxides (LP) in human cells (Jurkat) exposed or not to UVA+UVB irradiation. The evaluation of the amount of these ROS is obtained by using specific fluorescent probes and fluorescence flow cytometry analysis. This method offers a great sensitivity because the fluorescence of each cell is measured and numerous cells are evaluated. (10,000 cells per experimental condition).

Jurkat cells are cultivated in normal culture medium (one series not irradiated, one series irradiated; 3 different probes can be used). Cells are incubated in absence or in the presence of tested compounds (3 tested concentrations; triplicate recommended for UV exposed cultures and monoplicate for non-irradiated controls). Selected probes are added separately. For probes, see Carter (1994), J. Leukocyte Biol., 55, 253-258 (dihydrorhodamine (DHR), H₂0₂; dihydroethidium (DHE), O₂⁻); Makrigiorgos (1997), Free Rad. Biol. Med., 22, 93-100 (5-dodecanoylaminofluorescein (C1 1-fluor), lipid peroxides). After a 45 minutes incubation time a 37° C. (incorporation of probes in cells), cultures are exposed to a calibrated UVA+UVB irradiation or not (control). In presence of corresponding ROS probes HDR (H₂0₂) and HE (O₂⁻) are oxidized and become fluorescent whereas the fluorescence of the probe C1 1-fluor is decreased by oxidation. Tests are realized in triplicate; BHA (positive control) is used for the test validation. Fluorescence parameters are measured by flow cytometry on 10,000 cells for each sample. Results are expressed as percent of variation of ROS compared to control cultures.

### Example 7

### Lightening Properties

The following example evaluates the lightening properties of the compositions of the invention using in vitro assays of reconstructed epidermis. It is generally thought that the formulations of the compositions of the present invention effect the synthesis of melanin in the epidermis.

Compounds are topically applied on reconstituted epidermis containing melanocytes. After incubation, photographs of the epidermis surface are taken then cells are lysed and the amount of melanin is measured by photometry (optical density of the extract). Epidermis viability is controlled using the MTT assay. Compounds with lightening properties will reduce the OD475 nm.

An “n+I” series of 6 epidermis are prepared (n for the number of test compounds or concentrations). The tested compounds and the reference compound (kojic acid) are topically applied on the epidermis (2 mg cm⁻² for formulations). Each series corresponds to one treatment. A series remains untreated (control). Samples are incubated for 144 hours at 37° C., 5% CO2. Photographs are made of the epidermis. For 3 epidermis of each series, cell lysis and extraction of melanin are performed. Reading of the optical density occurs at 435 nm of each extract. The other 3 epidermis of each series are used for the evaluation of viability (option MTT assay). Results are expressed as variation (%) of melanin quantity compared to the negative control.

### Example 8

### Effects on Collagen and Elastin Synthesis

The following example evaluates the effects of compositions of the invention on collagen and elastin synthesis using an in vitro assay in co-cultures of reconstructed epidermis and human dermal fibroblasts. Applicants believe the compositions of the present invention stimulate collagen and elastin synthesis.

Because various formulations are not conducive to the use of monolayer culture of fibroblasts, effects of the compounds are evaluated using co-cultures of reconstituted epidermis and fibroblasts.

Culture inserts containing reconstituted human epidermis (RHE) are placed in culture wells containing monolayers of confluent normal human dermal fibroblasts (NHDF). Compounds are topically applied onto the RHE. Collagen synthesis is evaluated by the incorporation of radioactive proline, the main aminoacid in collagens. Because the amount of synthesised elastin is very low in these conditions, the effect of the compound is evaluated on the expression of the gene coding for elastin using the quantitative RT-PCR method.

Evaluation of collagen synthesis occurs as follows. NHDF are seeded in 24 wells culture plates and culture until confluence. An “n+ln series of 3 epidermis (n for the number of test compounds or concentrations) are prepared. Culture inserts containing RHE are placed in culture wells containing the NHDF. The tested compounds are topically applied on the epidermis: 3 epidermis treated with the formulation (2 mg cm⁻²); 3 epidermis treated with 100 μl of a solution of the active (same concentration as in the formulation); 3 epidermis not treated (control). Samples are incubated in NHDF medium containing 1% fetal calf serum, for 48 hours at 37° C., 5% CO₂. Some wells receive normal medium (unconditioned) alone (control) or containing vitamin C (reference compound). After 48 h of incubation, ³H-proline is added to the culture media and cells are further incubated for 24 h. Control of the viability of epidermis is assessed using the MTT test. Next occurring is extraction/solubilization of total proteins from cultures of fibroblasts and separate analysis of soluble and cell layer proteins followed by precipitation, filter-collection, and wash cycles. Quantification is by liquid scintillation.

Evaluation of elastin mRNA synthesis occurs as follows. NHDF is seeded in 24 wells culture plates and culture until confluence. An “n+1” series of 3 epidermis is prepared (n for the number of test compounds or concentrations). Culture inserts containing RHE are placed in culture wells containing the NHDF. The tested compounds are topically applied on the epidermis: 3 epidermis treated with the formulation (2 mg cm⁻²); 3 epidermis treated with 100 pl of a solution of the active (same concentration as in the formulation); 3 epidermis not treated (control). Incubation occurs in NHDF medium containing 1% fetal calf serum, for 48 hours at 37° C., 5% CO₂. Some wells receive normal medium (unconditioned) alone (control) or containing vitamin C (reference compound). Next occurring is extraction of total RNA, treatment with DNAse, then inactivation of DNAse. Analysis can be performed after pooling of the triplicates or on individual extracts. By reverse-transcription, cDNAs are quantified. Homogeneity of the preparations is controlled by comparison of Q-PCRs performed for the G3PDH marker (housekeeping gene). Q-PCRs are performed in duplicates using specific primers for sequences of G3PDH and elastin. Quantification of differential expressions is compared to G3PDH.

### Example 9

### Lightening Properties

The following example evaluates the lightening properties of the compositions of the invention using an vitro assay in cultures of melanocytes. Applicants believe the compositions of the invention effect the enzymatic activity of tyrosinase, a key enzyme of the synthesis of melanin. Optionally the effects of selected compounds on the quantity of melanin in normal human melanocytes in culture can be studied in a second step.

Step 1 studies the inhibition of tyrosinase activity. The tyrosinase extracted from mushroom is incubated in absence or in the presence of the tested compounds, then the enzyme substrate, L-DOPA, is added. The oxidation of the L-DOPA in DOPAquinone is measured by spectrophotometry. In the presence of an inhibitory compound, the optical density at 475 nm is decreased. Tyrosinase inhibition is measured as follows. Tyrosinase (mushroom) is incubated, for 10 minutes, in absence or in the presence of tested compounds (5 tested concentrations) or in the presence of kojic acid (positive control). Each experimental condition is performed in triplicate. L-DOPA is added and the samples are incubated for 1 h at 37° C. Optical density is measured at 450 nm. Results are expressed as percent of inhibition of the tyrosinase activity.

Step 2 studies cytotoxicity of the selected compounds in normal human melanocyte cultures. From this step, non-cytotoxic concentrations are selected. Cytoxicity is assessed as follows so as to further select compounds having an inhibitory effect on tyrosinase activity. Normal human melanocytes are seeded in culture medium containing the tested compounds (8 tested concentrations) or not (control). Incubation occurs at 37° C., 5% CO₂ for 72 hours. Viability assessment by using the MTT method.

Step 3 studies the effects on melanin synthesis in cultures of melanocytes. Melanocytes are incubated, during 240 hours, in absence and in the presence of the tested compounds prepared at 3 selected concentrations. The quantity of melanin in cells at the end of the incubation is measured by spectrophotometry. Melanin synthesis is assessed as follows. Normal human melanocytes are seeded and incubated until a 60-80% of confluence is reached. Culture medium is changed to medium containing the tested compounds (3 tested concentrations) or not (control), or containing kojic acid (positive control). Each experimental condition is performed in triplicate. Incubation is for 240 hours, followed by rinsing of cell monolayers and cell lysis. Melanin crystals are solubilized and the optical density read at 475 nm. Results are expressed as variation (%) of melanin quantity compared to the negative control.

### Example 10

### Effect on Dermal Extracellular Matrix Component Synthesis

The following example evaluates the effect of compositions of the invention on dermal extracellular matrix component synthesis, including fibroblast proliferation and collagen and glycosaminoglycan synthesis. Applicants believe the compositions of the invention effect fibroblast proliferation, proline-rich protein synthesis and glycosaminoglycan synthesis.

Assays are performed using radiolabelled precursor incorporation in neo-synthesised molecules.

A preliminary cytotoxicity is conducted in order to select non-cytotoxic concentrations used in the final assays. Normal human dermal fibroblasts are incubated, for 72 hours, in absence or in presence of the selected concentrations of the test compounds. During the last 24 hours of incubation radiolabelled precursor are added to the culture medium. Extraction and analysis of synthesized macromolecules allows quantifying cell growth, prolin-rich protein synthesis (mainly collagen) and glycosaminoglycan synthesis.

Cytotoxicity is determined as described above.

Fibroblast proliferation is determined as follows. Normal human fibroblasts are seeded in DMEM medium and incubated until a 30% of confluence is reached. Medium is changed to medium containing the test compounds prepared at non-cytotoxic concentrations, as determined above, or normal medium (control). Each experimental condition is performed in triplicate. Incubation occurs for 48 hours. ³H-thymidine is added to the culture medium. Incubation occurs for 24 hours. Extraction of macromolecules is performed by adapted techniques and thymidine incorporated in these macromolecule is measured by liquid scintillation. Results are expressed in variation of cell growth with regard to the control.

Proline incorporation is determined as follows. Normal human fibroblasts are seeded in DMEM medium and incubated until an 80% of confluence is reached. Medium is change to medium containing the test compounds prepared at non-cytotoxic concentrations, as determined above, or normal medium (control). Each experimental condition is performed in triplicate. Incubation occurs for 48 hours. ³H-proline is added to the culture medium. Incubation occurs for 24 hours. Extraction of macromolecules is performed by adapted techniques and proline incorporated in these macromolecule is measured by liquid scintillation. Results are expressed in variation of cell growth with regard to the control.

Glycosaminoglycan synthesis is determined as follows. Normal human fibroblasts are seeded in DMEM medium and incubated until a 80% of confluence is reached. Medium is change to medium containing the test compounds prepared at non-cytotoxic concentrations, as determined above, or normal medium (control). Each experimental condition is performed in triplicate. Incubation occurs for 48 hours. ³H-glucosamine is added to the culture medium. Incubation occurs for 24 hours. Extraction of macromolecules is performed by adapted techniques and glucosamine incorporated in these macromolecule is measured by liquid scintillation. Results are expressed in variation of cell growth with regard to the control.

### Example 11

### Protection of Cells from UV-Induced Lipid Peroxidation

The following example evaluates the effect of compositions of the invention on protection of cells from UV-induced lipid peroxidation in reconstructed epidermis. Applicants believe the compositions of the invention protect the epidermis against the deleterious effects induced by UV irradiation.

The tested compounds are applied on the upper side of reconstructed epidermis (SkinEthic, France). Epidermis are exposed to UVA+B irradiation, and after a 24 h incubation period, the content in lipid peroxides is evaluated using a biochemical assay. The assay is as follows. Human reconstructed epidermis (SkinEthic, 4 cm²) are cultivated in a specific medium. Tested compounds (triplicate, 2 mg cm²) or a reference compound are topically applied. Six epidermis are not treated. Incubation occurs for 18 hours at 37° C. Exposition to UVA+B occurs for treated epidermis and of 3 untreated epidermis (UV control). The other 3 untreated epidermis are kept in the dark (control-UV). Assessment of cell viability is performed on a 4 mm² punch from each epidermis. Dissociation of epidermis and extraction of lipids is performed with a chloroform/methanol mixture. Dosage of lipid peroxides is performed (Sigma kit). The protective effect is expressed as percentage of lipid peroxides compared to irradiated and non-irradiated control series.

### Example 11

In processing plants for poultry and animal products, it is customary to use various water treatment processes. such as a scalding tank, spray bath. final rinse and chill water tank. The scalding tank is used to dip poultry prior to the removal of feathers; other animals are dipped to remove the outer coating of fur or hair. The scalding process permits cross contamination and spread of pathogens. It is important for the safety of the human food supply to provide an additive that can be used in water treatments to inhibit the growth and spread of pathogens and deleterious bacteria. The ideal additive would not evaporate at boiling point temperatures, would not be destroyed by high temperatures and would not be bound by organic material, such as blood and feces and rendered useless.

The effect of PHB0020 on pathogenic, indicator, and spoilage populations of bacteria associated with broiler chicken carcasses in a poultry scald water application is determined in one embodiment of the present invention.

First, scalder water was collected from the overflow or entrance end of a 5 commercial poultry scalder. The water is sterilized or autoclaved to eliminate all populations of bacteria and bacterial spores to avoid interference during the study. The autoclaved scalder water is evaluated chemically and compared to raw scalder water to ensure that the organic material demand in raw and autoclaved scalder water is similar.

Next, sets of test tubes are prepared by adding 9 milliliters (ml) of sterilized scalder water to sterile polystyrene test tubes. One set is prepared as controls by adding 9 ml of sterilized scalder water to tubes. One set is prepared by adding 9 ml of sterilized scalder water and PHB0020 (the disinfectant) until the pH of 2.2 is achieved.

Each bacterium is exposed, one at a time, to the sterilized scalder water with PHB0020 sanitizer for approximately 2 minutes at approximately 130° F. (55° C.) to mimic scalding.

After the exposure period, one ml of the suspension was enumerated using the aerobic plate count method by pour plating and incubating at approximately 95° F. (35° C.) for 48 hours.

Table I below records microbial growth results in a scalder water project wherein sterilized water was heated to scalding temperatures of in a range of from approximately 120° F. (49° C.) to approximately 140° F. (60° C.), preferably to a temperature of approximately 130° F. (55° Q. Various concentrations of PHB0020 are added in a range between approximately 0.4 parts per million (ppm) to approximately 0.8 ppm. preferably at approximately 0.6 ppm and colonies of pathogens, indicator bacteria and spoilage bacteria are exposed to the treated scalder water.

TABLE I
Scalder Water Project

| | | | |
|---|---|---|---|
| | Colonies forming | | Growth after Exposure |
| Sample No.: | Bacteria Units | Log of Reduction | To Treated Scalder Water |

Bacteria: Control Salmonella typhimurium

| | | | |
|---|---|---|---|
| 1 | 430 | 2.633468 | negative (no growth) |
| 2 | 880 | 2.944483 | negative |
| 3 | 970 | 2.986772 | negative |
| 4 | 450 | 2.653213 | negative |
| 5 | 620 | 2.792392 | negative |
| 6 | 700 | 2.845098 | negative |
| 7 | 1140 | 3.056905 | negative |
| 8 | 620 | 2.792392 | negative |
| 9 | 580 | 2.763428 | negative |

Bacteria: Control Staphylococcus aureus

| | | | |
|---|---|---|---|
| 1 | 530 | 2.724276 | negative (no growth) |
| 2 | 550 | 2.740363 | one (1) colony growing |
| 3 | 580 | 2.763428 | negative |
| 4 | 500 | 2.698970 | negative |
| 5 | 540 | 2.732394 | negative |
| 6 | 420 | 2.623249 | negative |
| 7 | 530 | 2.724276 | negative |
| 8 | 480 | 2.681241 | one (1) colony growing |
| 9 | 470 | 2.672098 | negative |

Bacteria: Control Pseudomonas fluorescens

| | | | |
|---|---|---|---|
| 1 | 540 | 2.73234 | negative |
| 2 | 880 | 2.944483 | negative |
| 3 | 790 | 2.897627 | negative |
| 4 | 620 | 2.792392 | negative |
| 5 | 1120 | 3.049218 | negative |
| 6 | 790 | 2.897627 | one (1) colony growing |
| 7 | 5200 | 3.716003 | negative |
| 8 | 1360 | 3.133539 | negative |
| 9 | 1040 | 3.017033 | negative |

Bacteria: Control Listeria monocytogenes

| | | | |
|---|---|---|---|
| 1 | 1720 | 3.235528 | five (5) colonies growing |
| 2 | 1840 | 6.264848 | six (6) colonies growing |
| 3 | 1440 | 3.158362 | negative (no growth) |
| 4 | 1820 | 3.260071 | five (5) colonies growing |
| 5 | 1440 | 3.158362 | one (1) colony growing |
| 6 | 1880 | 3.274158 | negative |
| 7 | 1720 | 3.235528 | negative |
| 8 | 1720 | 3.235528 | negative |
| 9 | 1740 | 3.240549 | negative |

Bacteria: Control Shewanella putrefaciens

| | | | |
|---|---|---|---|
| 1 | 50 | 1.698970 | negative (no growth) |
| 2 | 50 | 1.698970 | negative |
| 3 | 60 | 1.778151 | negative |
| 4 | 20 | 1.301030 | negative |
| 5 | 50 | 1.698970 | negative |
| 6 | 70 | 1.845098 | negative |
| 7 | 80 | 1.903090 | negative |
| 8 | 20 | 1.301030 | negative |
| 9 | 30 | 1.477121 | negative |

Bacteria: Control Escherichia coli

| | | | |
|---|---|---|---|
| 1 | 15100000 | 7.178977 | 460 colonies growing |
| 2 | 12900000 | 7.110590 | negative (no growth) |
| 3 | 13300000 | 7.123852 | 32 colonies growing |
| 4 | 12200000 | 7.086360 | 1170 colonies growing |
| 5 | 13400000 | 7.127105 | 4700 colonies growing |
| 6 | 12200000 | 7.086360 | 57 colonies growing |
| 7 | 14200000 | 7.152288 | 900 colonies growing |
| 8 | 13600000 | 7.133539 | 410 colonies growing |
| 9 | 7600000 | 6.880814 | 37 colonies growing |

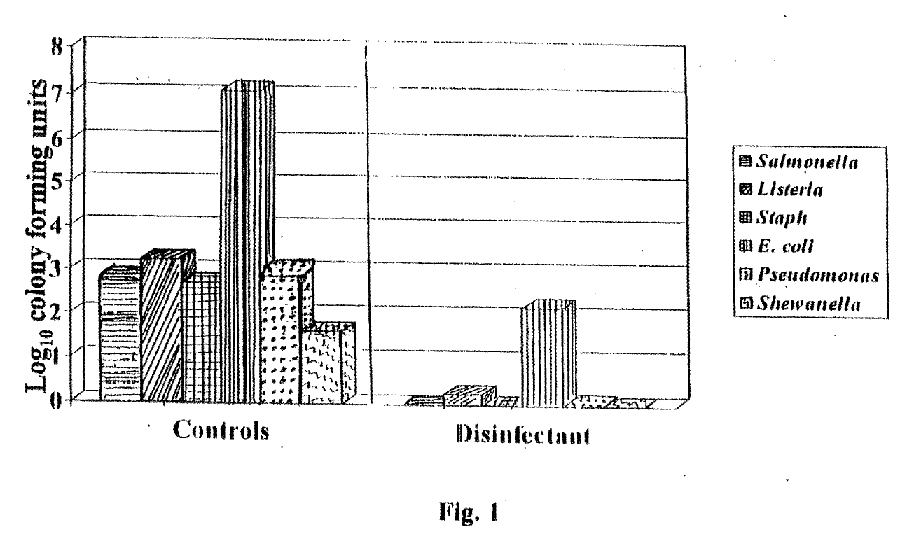

Referring now to FIG. 1, the graph shows the effect of PHB0020 on pathogenic and spoilage bacteria identified in the table above. The graph is divided in two sections, on the left is the control showing the logarithm of colony forming units for each bacterium and on the right is the graph of colony forming units after each bacterium is exposed for 2 minutes to scalder water treated with PHB00200. The graph shows that Listeria, a gram-positive bacterium, is hard to kill and E coli, a “veil’ prolific bacter”, has the highest reduction after a 2 minute exposure.

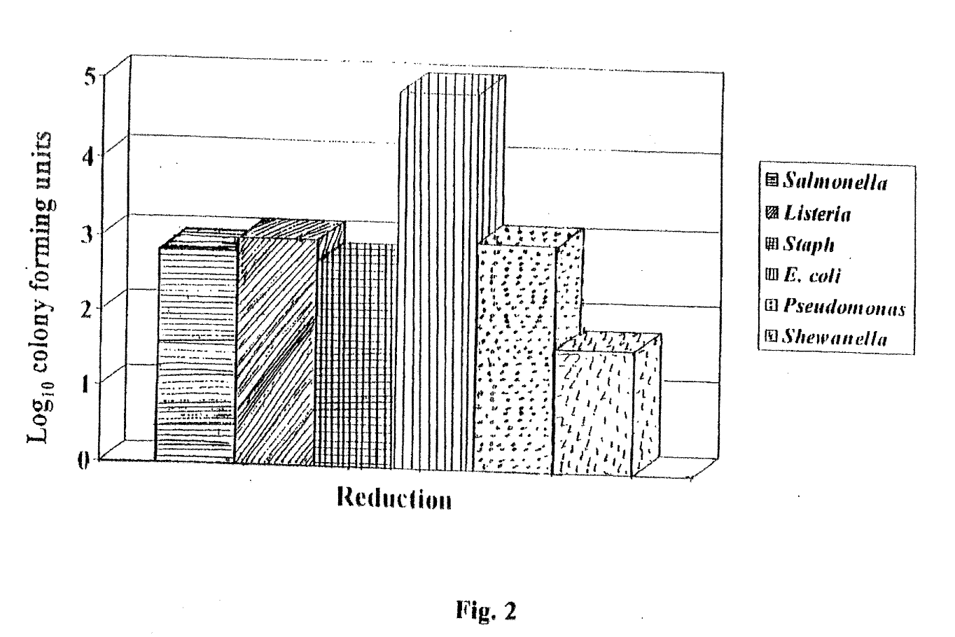

In FIG. 2, the graph shows the logarithm of the reduction of bacterial levels for each bacterium. In most cases the log of colony forming units is less than three, with the most prolific bacterium, E coli having a log of less than five.

Thus, PHB0020 functions as an antimicrobial agent, disinfectant, or sanitizer and is extremely effective for eliminating populations of pathogenic, indicator and spoilage bacteria in commercial scalder water under industrial scalding conditions. PHB0020 is an effective means for controlling bacteria in scalder water and may be used for controlling cross-contamination during scalding. Disinfection of poultry scalder water is crucial because it is the first area within the plant in which birds are immersed in a common bath wherein bacteria can be transferred from bird to bird.

What is claimed is:

1. An anti-wrinkle skin care composition comprising an effective amount of:
a mixture comprising PHB0020 containing metallic ions, a standard dermal cream; and
water to form a cream that is effectively delivered to the subdermal layer of the skin.

2. The composition of claim 1, wherein the metallic ions are selected from the group consisting of copper, magnesium, silver and zinc.

3. The composition of claim 2, wherein the metallic ions are copper ions.

4. The anti-wrinkle skin care composition of claim 1, further comprising a pH modifier to adjust the pH of the composition to a range between approximately 2.75 and approximately 3.20.

5. The anti-wrinkle skin care composition of claim 4, wherein the pH of the composition is approximately 3.0.

6. A skin care composition for bur relief comprising an effective amount of:
a mixture comprising PHB0020 containing metallic ions, a standard dermal cream: and
water to form a product that effectively relieves the pain of a burning sensation and heals the damage to epidermal layers caused by burns.

7. The composition of claim 6, wherein the metallic ions are selected from the group consisting of copper, magnesium, silver and zinc.

8. The composition of claim 7, wherein the metallic ions are copper ions.

9. The skin care composition of claim 6, further comprising a pH modifier to adjust the pH of the composition to a range between approximately 0.50 and approximately 4.0.

10. The skin care composition of claim 9, wherein the pH of the composition is approximately 1.5.

11. A topical skin care composition for insect bites comprising an effective amount of:
a mixture comprising PHB0020 containing metallic ions. a standard dermal cream: and
water to form a product that effectively relieves the pain of an insect bite and heals the damage to epidermal layers caused by said bites.

12. The composition of claim 11. wherein the metallic ions are selected from the group consisting of copper. magnesium, silver and zinc.

13. The composition of claim 12, wherein the metallic ions are copper ions.

14. The skin care composition of claim 11, further comprising a pH modifier to adjust the pH of the composition to a range between approximately 0.50 and approximately 4.0.

15. The skin care composition of claim 14, wherein the pH of the composition is approximately 1.5.

16. A method of preparing a skin care composition that is useful for treating, preventing or ameliorating environmental or age-related damage or deterioration of the skin comprising the steps of:
mixing PHB0020 containing metallic ions with deionized water;
adding standard cosmetological and dermatological ingredients: and
stirring until completely blended and metallic ions are uniformly suspended.

17. The method of claim 16, wherein the metallic ions are selected from the group consisting of copper, magnesium, silver and zinc.

18. The method of claim 17, wherein the metallic ions are copper ions.
